SEQUENCE LISTING (1 sequence)

SEQ ID NO: 1 - DNA, 32 bp, artificial sequence
  PCR-primer
ntttgtctcgagtgttttcactcgagactttn

(19) United States
(12) Patent Application Publication
(10) Pub. No.: US 2006/0057571 A1
(43) Pub. Date: Mar. 16, 2006

(54) Methods for detecting nucleic acids while using solid phase bound primers and cleaving the products of a cyclic amplification reaction (trap release primer amplification (tramp)

(76) Inventor: Reinald Repp, Fulda (DE)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/500,601

(22) PCT Filed: Dec. 31, 2002
(86) PCT No.: PCT/DE02/04754

§ 371 Date: Jun. 10, 2005

(30) Foreign Application Priority Data
Dec. 31, 2001 (DE) 101-64-219.9

(51) Int. Cl. C12Q 1/68; C12P 19/34

(52) U.S. Cl. 435/6; 435/91.2

(57) ABSTRACT

The invention relates to methods for detecting nucleic acids with the aid of an amplification reaction whereby involving a novel structure of solid phase bound starter molecules (primers) with a cyclic amplification reaction on the solid phase and a transformation of the amplification products into the aqueous phase. The solid phase bound primers, by binding to their specific target sequence, undergo a conformation change that generates a cleaving site. Newly synthesized reaction products can be cleaved and transformed into the aqueous phase via this cleaving site. In the following reaction cycle, these reaction products are provided as additional matrices (templates) whereby enabling an exponential product multiplication.

The present invention relates to methods for detecting nucleic acids by means of an amplification reaction, involving a novel combination of solid-phase-bound starter molecules (primers) with a cyclic amplification reaction on the solid phase and transfer of the amplification products into the aqueous phase. The fact that the primers are coupled to a solid phase means that important disadvantages of the traditional polymerase chain reaction (PCR), such as the formation of primer dimers and the obtaining of false-positive results by product-product interactions are profoundly reduced. To achieve an exponential multiplication of product, however, the PCR products which are newly synthesized in each cycle must be released from the solid phase. This is achieved by a conformation change within the primers, which is triggered by the binding to the target sequence to be recognized and which permits selective cleavage, for example by means of a restriction enzyme.

The polymerase chain reaction (PCR) is a method for the exponential multiplication of specific DNA segments. The course of the reaction which is generally used for this purpose is very simple since all of the reagents and starter molecules are present in the reaction mixture and tolerate the absolute temperatures and the temperature changes which are required for the cyclic course of the reaction. In this manner, the three-step sequence which is repeated many times, generally 25-50 times, viz. the denaturation of double-stranded DNA molecules to give single strands, the binding of the matching single strands to the complementary starter molecules (annealing phase) and the enzymatic elongation of the starter molecules (primers) along the bound complementary target sequence (elongation), allows a multiplication of the target sequence to be achieved, resulting in high sensitivity. Under optimal conditions, the molecules of the target sequence are doubled in each of these cycles. Under practice conditions, however, this theoretical efficiency is achieved during the first reaction cycles at most (exponential PCR phase). In later cycles, negative effects which reduce the efficiency of the PCR amplification and which lead to asymptotic approximation of the number of target sequence molecules to a reaction-specific limit beyond which no further multiplication takes place, are observed increasingly during the later cycles. The reason for this is firstly the increasing consumption of the resources employed (enzyme activity, primers, nucleotide triphosphates), but more importantly increasingly occurring interactions between the nucleic acids present in the reaction mixture. Those which are of importance in this context are, for example, the unspecific binding of primers, which may lead to a linear “background amplification” or indeed to false-positive results when a second primer binds in the vicinity at the counter strand. These effects play a role in particular in so-called multiplex PCR reactions, where a substantial number of primers for assessing a plurality of target sequences is employed in a single reaction mix. A further problem, which also gains in importance when carrying out a multiplex PCR, is the formation of primer dimers. Here, the starter molecules (primers) employed interact with one another, resulting in a DNA chain elongation along the second primer sequence. Owing to their small size, such primer dimers are amplified very efficiently, so that the resources employed in the PCR may already be consumed before a discernable amplification of the target sequence has taken place.

After many PCR cycles have been performed, competitive effects between the two strands of the PCR product and the primers may increasingly adversely affect the amplification efficiency. Here, the amplification of the target sequence is adversely affected either still before, or during, chain elongation owing to the complete or partial pairing of the two DNA strands of the target sequence. In addition to the adverse effect on the amplification efficiency, the interaction between DNA molecules which have already been newly synthesized in the PCR may lead to the formation of unspecific products or to false-positive results. This is the case when the 3′ ends of these DNA single strands can interact with one another. Again, the risk is greater in multiplex reactions since the risk that DNA strands with 3′ ends which are homologous to one another and which, after pairing with one another and completing to DNA double strands, may be amplified exponentially in the subsequent cycles by the DNA polymerase employed, which eventually gives rise to false-positive or artificial products, increases with each target sequence to be amplified. Although several of the described mechanisms which adversely effect the PCR amplification have graver effects in multiplex reactions, multiplex reactions are increasingly being employed since they make it possible to assess a multiplicity of target sequences in a single reaction. Since the number of known nucleic acid sequences is rising drastically, it can be expected that this trend is going to last.

An alternative which can be used for the parallel assessing of the many nucleic acid target sequences is provided by what are known as “gene chips”. However, they are much less sensitive than PCR since they detect the nucleic acids via specific hybridization reactions and no enzymatic amplification of the target sequences takes place.

The problem of the present invention thus consists in increasing the number of the target sequences which can be assessed in parallel in a PCR reaction, by largely eliminating the above-described adverse factors which are increasingly observed in multiplex reactions, such as the formation of primer dimers and the generation of false-positive results as the consequence of unspecific product-product interactions via complementary 3′ ends, while simultaneously enabling an exponential amplification of the target sequences.

This problem is solved in accordance with the invention by a method for the amplification of nucleic acids, comprising the following steps:

i) providing nucleic acids to be amplified,

ii) carrying out the amplification reaction using at least two primers, of which at least one is coupled with a solid phase,

iii) transfer of the reaction products into the aqueous phase,

iv) detecting of the amplification products formed in ii) via fluorescent-optical, radioactive, chemical, chromatographic or any other methods for detecting nucleic acids,

characterized in that
a) at least one of the primers employed for the amplification reaction comprises, in 5′-3′ direction, the following elements: coupling site for the solid phase, a first target-sequence-specific segment, a double-strand-specific cleavage site, an optional sequence which may serve for product detection, reamplification in a subsequent conventional PCR reaction (nested PCR), as primer binding site for a sequence reaction or for further functions, optional further sequence segments which serve for the stabilization of the double-strand-specific cleavage site, and a second sequence which is specific for the nucleic acid to be amplified,
b) de-novo-synthesized DNA molecules are transferred into the aqueous phase by cleavage with a restriction enzyme either still during the de-novo synthesis or after the PCR amplification cycles.

While coupling PCR primers with a solid phase can prevent the formation of primer dimers, it simultaneously no longer enables an exponential product multiplication since only those molecules of the target sequence which are already present in the aqueous phase prior to the beginning of the reaction will hybridize with the solid-phase-bound primers and will initiate a de-novo DNA synthesis by primer elongation. Since the newly synthesized DNA which has been formed owing to the elongation of the solid-phase-bound primers remains bound to the solid phase, it is only capable of binding to complementary primer sequences in a very limited fashion in the immediate vicinity and is therefore almost not at all available as template for a further DNA synthesis. That is to say that the pool of the template strains of the target sequence which makes possible a de-novo DNA synthesis is not enlarged substantially during the reaction cycles, resulting in just linear amplification kinetics (Adessi et al. (2000), Nucleic Acids Research 28.20, e87).

An essential prerequisite for an exponential amplification of the target sequences is the transfer of the DNA molecules synthesized in the PCR into the pool of potential DNA template strands which, when binding complementary primers, again enable a de-novo DNA synthesis. To this end, the two DNA strands of the target sequence must be able to hybridize freely with complementary primers after each PCR cycle. This can be achieved by transferring the newly synthesized DNA into the aqueous phase without adversely affecting the binding of the single-stranded primers of the solid phase. Restriction enzymes may be used for this purpose since they restrict double-stranded DNA selectively at defined recognition sequences. When the double-stranded DNA is cleaved from the solid phase, both strands can hybridize with the complementary primers after the PCR denaturation step. The pool of potential DNA template strands can double in each cycle; an exponential amplification of the target sequence is possible. However, a decisive prerequisite for this is that the recognition sequence for restriction enzymes is not destroyed by the restriction, i.e. the enzymes used must restrict at the 5′ end of the recognition sequence or further upstream in 5′ direction. Only in this way is the recognition sequence retained and can mediate the cleavage of double-stranded DNA molecules from the solid phase in the following cycles. A cleavage site further in the 5′ direction is more advantageous than directly at the 5′ end of the recognition sequence since restriction enzymes restrict less efficiently when their recognition sequence is located directly at the end of a double-stranded DNA molecule and when a few further terminal base pairings are lacking.

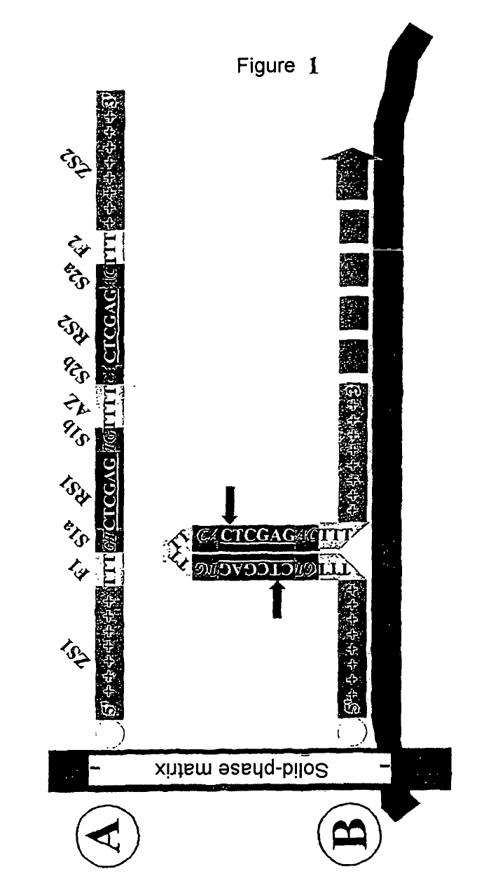

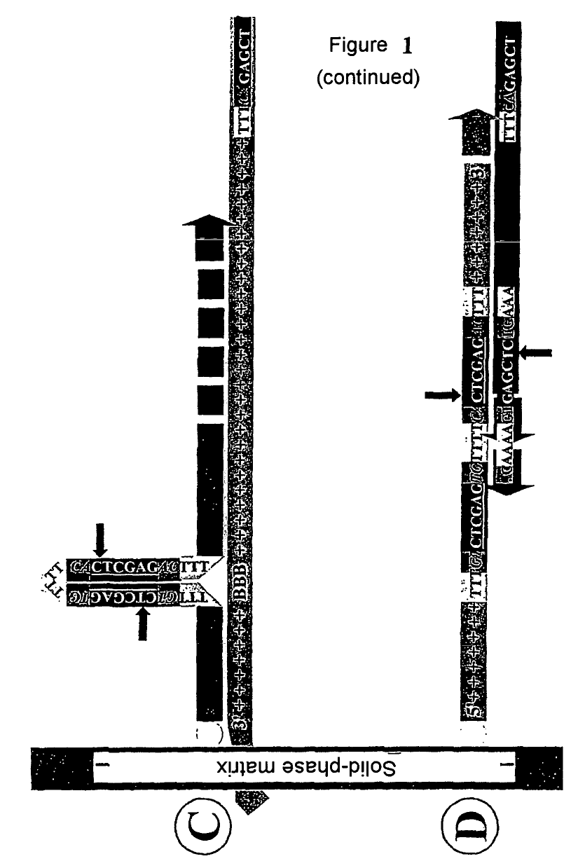

The TR-PCR can be carried out analogously to conventional PCR when the restriction enzyme used tolerates the temperatures and buffer conditions employed in the PCR. The solid-phase coupling of the PCR primers can be effected directly or indirectly to the reaction vessel itself including plastic containers and glass capillaries, or the primers are coupled with a carrier which is then introduced into the reaction vessel. The decisive steps in the course of the reaction are shown schematically in FIG. 1:

segment “A” of FIG. 1 shows the various domains of the PCR primers. A structure (shown as a circle) which mediates the binding of the oligonucleotide to the solid phase is located at the 5′ end. This is followed, in 3′ direction, by a first domain which is complementary to the target sequence (ZS1). The length and sequence of this domain is designed to suit the annealing temperature used in the PCR reaction. The specific binding between the primer and the nucleic acid target sequence to be amplified is to take place at this temperature. An analogous domain which binds to the target sequence is located at the outermost 3′ end of the primers (ZS2). These two primer domains which bind to the target sequence are separated by interposed nucleotides which, when the primer binds to the target sequence, undergo a conformation change and thus generate a double-stranded restriction site. From 5′ to 3′ direction, this region comprises the following functional segments: flexibilization domain 1 (F1, shown here for example by 3 “T” nucleotides), stabilization range 1a (S1a, nucleotide names in italics), restriction site sequence 1 (RS1, nucleotide names underlined), stabilization region 1b (S1b), spacer AZ (this centrally located spacer optimally consists of 4 “T” nucleotides since these mediate a complete change in direction within the 3-dimensional molecule structure and thus facilitate the development of a hairpin structure), stabilization domain 2 (S2b), restriction site sequence 2 (RS2), stabilization domain 2a (S2a), flexibilization domain 2 (F2). The nucleotide sequence of the stabilization and restriction site domains 1 is reverse-complementary to that of the analogous domains number 2. This enables the formation of a hairpin structure within the primer when the temperature falls short of a certain limit. In addition to the restriction site sequence, length and sequence of the stabilization domains determine this limit temperature. It is chosen in such a way that it is below all of the temperatures set during the course of the PCR reaction. This ensures that, during the PCR reaction, the solid-phase-bound primers exist in linear structure and do not develop hairpin structures as long as the primers do not bind to the target sequence with their two terminal specific domains. Apart from the two domains which bind to the target sequence (ZS1 and ZS2) and the restriction site sequences (RS1 and RS2), all the other primer segments only serve as aids for achieving a specific structure as a function of the binding to the target sequence, and therefore need not be present at all times.

Part “B” of the figure shows the structures as they develop after the binding of a primer to its target sequence (“---” symbolizes the negative polarity chosen in the example). owing to the binding of the two specific primer domains to the target sequence, these two primer segments come into the immediate vicinity of one another. This stabilizes the potential hairpin structure which the primer region can form between the terminal, target-sequence-specific domains. As a consequence, the hairpin structure can also be formed at temperatures higher than is the case with the single-stranded primer molecule, after the primer has been bound to its target sequence. By choosing the sequence of the primer segments referred to as “stabilization domains” and other parameters which have an effect on the melting point of the hairpin structure, such as, for example, the distance between the two primer binding domains on the target sequence, the limit temperature between linear primer structure in the native state and development of a hairpin structure after the primer has been bound to its target sequence is set in such a way that it is exceeded slightly during the lowest temperature level of the PCR reaction, i.e. only primers which are bound to their target sequence develop a hairpin structure during the PCR reaction while single-stranded primer molecules remain in linear form during the entire PCR reaction.

The section marked “BBB” in the target sequence must bridge the distance between the two strands of the hairpin structure, in accordance with the distance required for the formation of the hydrogen bonds between the nucleotides involved in the hairpin structure. Here, no nucleotides which are capable of interacting with the “flexibilization domains” of the primers (F1, F2) should be present in the target sequence. Since, in the present example, “T” nucleotides were chosen for the “flexibilization domains”, no “A” nucleotides should be present in the spacer (BBB) of the target sequence. Owing to the development of the hairpin structure, the two restriction domains of the PCR primer which are reverse-complementary to one another anneal to form a DNA double strand so that the corresponding restriction enzyme is capable of cleaving (cleavage sites are shown by black arrows). If a heat-resistant restriction enzyme with sufficient activity under the PCR buffer and temperature conditions is used, this enzyme may simply be added to the PCR mix. The figure shows the sequence of the heat-resistant restriction enzyme TliI. In their linear single-stranded structure, which prevails as long as no binding to the target sequence has taken place, the solid-phase-bound primers are not cleaved. However, once the primer has bound to its target sequence, the double-stranded recognition domain for the corresponding restriction enzyme forms via the development of a “hairpin conformation”. The primer is cleaved in its central segment by the restriction enzyme, while strand elongation takes place at the 3′ end of the primer along the target sequence. The newly synthesized DNA strand together with the 3′ region of the PCR primer is thereby cleaved from the solid phase coupling site. In the PCR denaturation phase which follows, in which the hydrogen bonds of the DNA double strand are separated at high temperature, all of the newly synthesized DNA single strands are set free and are again able to bind to complementary primer sequences in the following PCR cycle. This is shown in segment “C”.

As the product of the first amplification cycle, the plus-stranded template strand shown in segment “C” comprises, at its 5′ end, sequence segments which originate from a PCR primer and which include portions of the restriction cleavage site. In this example of a cycle, the complementary minus-strand of the target sequence is complemented to give the double strand up to these bases. What is important in this context is that a DNA polymerase is used in the PCR which adds no additional nucleotides, in this case as an adenosine residue, at the 3′ end of the newly synthesized strand, unless precisely this base corresponds to the sequence required for complementing the restriction cleavage site.

Segment “D” shows an example of how the minus strand synthesized in segment “C”, in turn, can act as template for the synthesis of another plus strand. The minus strand already comprises, at the two ends, sequence segments which originate from PCR primers. After a plus-stranded PCR primer has bound to the target-sequence-specific domain at the 3′ end, its own 3′ end can be elongated along the primer sequence (arrows pointing left) while the synthesis of the new plus strand takes place simultaneously (arrow pointing right). This regenerates the restriction cleavage site, which had previously existed in part only. The product of this PCR run can also be cleaved without the formation of a hairpin structure within the primer. The formation of a restriction cleavage site within the PCR primer by formation of a hairpin structure owing to specific binding to the target sequence thus ensures that cleavage of the newly synthesized PCR products can also occur if the de-novo synthesis takes place at the native starting DNA (segment “B) or at the product of the first amplification reaction (segment “C”). All further amplification products comprise, at both ends, part-sequences which already originate from PCR primers and which can be elongated in a linear fashion to give complete restriction cleavage sites (segment “D”).

A prerequisite of the course of the reaction shown is that the target sequence to be amplified does not comprise the same restriction cleavage sites as are used within the PCR Primers. Owing to the use of rarely cleaving restriction enzymes, no substantial limitations apply.

Trap release primer amplification (TRAmp) makes it possible to increase the number of simultaneously detectable target sequences in multiplex PCR reaction mixes since undesired primer-primer interactions are largely eliminated. The fact that a binding to two specific sequence segments of each primer is required for the amplification and cleavage of the products, at least during first amplification cycles, increases the specificity of the reaction. Moreover, a large number of PCR primers may be coupled to a solid phase in ready-made form. If the primers are coupled to a reaction vessel or a template to be added to the reaction, multiplex reactions are simplified considerably since the pipetting steps required for making the primer combinations are dispensed with.

1. A method for detecting nucleic acids using solid-phase-bound primers which, by binding to their specific target sequence, undergo a conformation change which enables the selective cleavage of the products of the cyclic amplification reaction (trap release primer amplification (TRAmp)), comprising the steps
i) providing nucleic acids to be amplified,
ii) carrying out the amplification reaction using at least two primers, of which at least one is coupled with a solid phase,
iii) generation of a cleavage site within the solid-phase-bound primers by a conformation change resulting from binding to the primer-specific target sequence,
iv) releasing the reaction products into the aqueous phase by cleavage within the cleavage site generated in ii) by conformation change, e.g. with the aid of a restriction enzyme,
v) detecting of the amplification products formed in ii) via fluorescent-optical, radioactive, chemical, chromatographic or any other methods for detecting nucleic acids,
characterized in that
a) at least one of the primers employed for the amplification reaction comprises, in 5′-3′ direction, the following elements: coupling site for the solid phase, a first target-sequence-specific segment, a cleavage site generable by conformation change, an optional sequence which may serve for product detection, reamplification in a subsequent conventional PCR reaction (nested PCR), as primer binding site for a sequence reaction or for further functions, optional further sequence segments which serve for the stabilization of the double-strand-specific cleavage site, and a second sequence which is specific for the nucleic acid to be amplified,
b) de-novo-synthesized DNA molecules are transferred into the aqueous phase by cleavage with a restriction enzyme either still during the de-novo synthesis or after the PCR amplification cycles.
